(19) United States
(12) Patent Application Publication
(10) Pub. No.: US 2015/0282723 A1
(43) Pub. Date: Oct. 8, 2015

(54) Device and Method for Detecting and Signalling a Stress State of a Person

(71) Applicant: XOTOX TOOLS AG, Küsnacht (CH)

(72) Inventors: Philipp Bachmann, Kusnacht (CH); Rolf Fritzsche, Hinwil (CH)

(73) Assignee: xotox tools ag, Kusnacht (CH)

(21) Appl. No.: 14/435,586

(22) PCT Filed: Sep. 20, 2013
(86) PCT No.: PCT/EP2013/069647

§ 371 Date: Apr. 14, 2015

(30) Foreign Application Priority Data
Oct. 16, 2012 (EP) 12188701.2

(51) Int. Cl. A61B 5/024; A61B 5/16; A61B 5/00

(52) U.S. Cl. CPC A61B 5/02405; A61B 5/7278; A61B 5/165; A61B 5/7282

(57) ABSTRACT

For easy determination of the current stress condition of a person, a device is proposed that detects the pulse rate and therefrom additionally determines the heart rate variability. In addition, at least one parameter related to the history of one of the two above mentioned values shall be used. Preferably, the deviation of the cardiac frequency and of the heart rate variability from a reference value is integrated and used as an additional stress indicator.

### TECHNICAL FIELD

The invention relates to a device and a method for detecting and reporting of a stress condition of a person.

### PRIOR ART

Using the pulse rate or the heart rate variability for the detection of stress conditions has been known for a long time. Thereby, the interval between two heart beats is defined—in the sense of the present invention—as the time between the onsets of two contractions of the cardiac chambers. This onset of the chamber contraction shows up in the electrocardiogram (ECG) as the R wave. The distance between two R waves is usually denoted as RR interval. After averaging over a defined number of RR intervals, the heart rate can be determined by calculation. The individual values of the RR intervals vary around the mean value thus obtained. Thereby the variations can change from beat to beat. The variation is usually denoted as heart rate variability (HRV). In principle, the heart rate can also be determined by a pressure measurement carried out on an artery.

Physiologically, the heart rate variability (HRV) is related to the ability of the human organism to adapt the rate of the cardiac rhythm. Variations of the heart rate, i.e. variations of the temporal interval between two heart beats, can occur in a resting state, in which case they are mostly spontaneous, but also upon specific variations of the surrounding conditions, e.g. under stress. A healthy organism continuously adapts the heart beat rate to the current conditions via physiological regulation pathways of the vegetative nervous system. Therefore, physical or psychological stress usually results in an increase of the heart frequency which ordinarily decreases again upon relief and relaxation. Thereby, a good adaptability to stress results in a higher variability of the heart rate. Under chronic stress burden, the adaptability is reduced. In this respect, it is known that the heart rate variability taken by itself already provides a certain—albeit still very unreliable—indicator for the current stress burden and the ability of a person to cope with stress.

Several methods for determining the stress condition of a person have been proposed in the prior art, including the proposal to use further measurement parameters in addition to the pulse rate. Thus, DE 103 19 361 A1 proposes to use the pulse wave latency in addition to the heart rate variability.

Regarding the analysis of the heart rate variability, reference is made to DE 100 06 154 A1 ,DE 10 2006 039 957 A1, and also to DE 10 2008 030 956 A1 and EP 1 156 851 B1, in which the person skilled in the art can find various determination methods.

From EP-2 316 333 A1 there is known a device and a method in which a state quantity is calculated which is a function of the current heart rate and of the current heart rate variability, preferably a linear combination of the current heart rate P and of the current heart rate variability. Moreover, in the mentioned publication, it is proposed to provide the state function with at least one correcting value that includes the history of the person within at least the past 0.5 hours.

This approach appears quite suitable here. However, there results a further optimization problem which can be described by the fact that, on the one hand particularly for comparing different persons—it is necessary to introduce normalizations, but that on the other hand it is also necessary to take into account individual differences.

### DESCRIPTION OF THE INVENTION

The object of the invention is to provide a device and a corresponding method for detecting and reporting of a stress condition of a person with an increased reliability as compared to the state of the art, and in which, on the one hand—particularly for comparing different persons—normalizations can be introduced, but in which, on the other hand, individual differences are also taken into account.

The object of the invention is achieved—according to a first aspect—by a method according to claim 1. At the outset, the features of the invention have the result that, to a first approximation, namely in the first processing window, i.e. in a first time interval T₁ or across a predetermined number of pulse beats, a first approximation result is obtained that can already be processed. For this purpose, one may consider, in particular, age related values that provide—preferably for the respective age groups—typical tabulated values of the minimum and the maximum values of the pulse rate and the HRV value. The subsequent processing windows then can—according to experimental studies—provide results that are improved, particularly if the person has characteristics that deviate from the tabulated values of the minimum and the maximum values of the pulse rate and the HRV value, regardless of whether this is due to reasons of e.g. a particularly good or particularly bad training condition or whether it is also due to high or low stress values in the near or medium term history. It should be noted that recording of the pulse is possible anywhere on the body and that the pulse can in principle be measured optically, acoustically or kinesthetically, and converted to electrical signals. Also, a direct measurement of electrical signals for the purpose of pulse measurement is possible according to the invention.

According to the method for detecting and reporting of a stress condition of a person according to the present invention, the following steps are carried out:

Within the time interval, or for a predetermined number of pulse beats, the data of the current pulse frequency P and of the current heart rate variability HRV are continuously acquired and processed. Thereby, a value for the stress index is separately calculated for the pulse rate and for the heart rate variability, and subsequently the two values are added—using weighting factors. In this step the values of the pulse rate and of the heart rate variability are normalized with respect to tabulated extremal values Pₘₐₓ, Pₘᵢₙ, HRVₘₐₓ and HRVₘᵢₙ, which extremal values have preferably been sorted according to age. In this manner a first potentially already useful—value for the stress index is determined.

Subsequently, however, one determines for said time interval, or for said predetermined number of pulse beats, whether the individual extremal values Pₘₐₓ, Pₘᵢₙ, HRVₘₐₓ ^{and HRV}ₘᵢₙ are different from the previously adopted values. For the subsequent time interval or for the subsequent measurement of predetermined pulse beats, for which neither the length of the time interval nor the number of the pulse beats necessarily needs to be identical to that of the first measurement, and which can also be significantly later, i.e. after a temporal delay of up to several hours, either the earlier extremal values or the newly determined extremal values are used, depending on which values are more extremal. The calculation of the new values for the stress index then occurs basically in an analogous manner as the above described first measurement, i.e. a value of the stress index is calculated by continuously and separately determining the stress index values for the pulse rate and for the heart rate variability and subsequently adding the two values with weighting factors. The time interval lies typically in the range of 100 s to 1000 s—preferably of 300 s to 500 s—but it can also be about one order of magnitude (factor 10) smaller or larger. The predetermined number of pulse beats lies between 50 and 500, preferably 100, but it can be larger, in particular, by about one to two orders of magnitude. It should be emphasized that the subsequent measurements may temporally overlap, in which case at the onset of the new series of measurements clearly only those extremal values can be used that have previously occurred. This “moving-window” method can be appropriate if massive computing power is available and a very rapid result shall be achieved.

Advantageously, the method can be carried out if the normalizations are each carried out by means of a normalization value at the first, table-related

P_{z}=Pₘᵢₙ+a*(Pₘₐₓ−Pₘᵢₙ)

HRV_{z}=HRVₘᵢₙ+b*(HRVₘₐₓ−HRVₘᵢₙ)

and the calculations of the summands of the stress value SI are each carried out according to

SI_{P}=(P_{d1}−P_{z})/(Pₘₐₓ−P_{z}) if P_{d1}>P_{z}

SI_{P}=(P_{d1}−P_{z})/(P_{z}−Pₘᵢₙ) if Pa_{d1}<P_{z}

SI_{HRV}=−(HRV_{d1}−HRV_{z})/(HRVₘₐₓ−HRV_{z}) if HRV_{d1}>HRV_{z}

SI_{HRV}=−(HRV_{d1}−HRV_{z})/(HRV_{z}−HRVₘᵢₙ) if HRV_{d1}<HRV_{z},

The further windows are then advantageously calculated according to

SI_{P}=(P_{dx}−P_{z})/(Pₘₐₓ−P_{z}) if P_{dx}>P_{z}

SI_{P}=(P_{dx}−P_{z})/(P_{z}−Pₘᵢₙ) if P_{dx}<P_{z}

SI_{HRV}=−(HRV_{dx}−HRV_{z})/(HRVₘₐₓ−HRV_{z}) if HRV_{dx}>HRV_{z}

SI_{HRV}=−(HRV_{dx}−HRV_{z})/(HRV_{z}−HRVₘᵢₙ) if HRV_{dx}<HRV_{z},

It has been proven advantageous if the values a for the processing of the pulse rate are selected in the range between 0.2 and 0.3, advantageously as 0.25 and the values b for the processing of the heart rate variability between 0.33 to 0.66, advantageously as 0.5.

Depending on the application the method can be calibrated in the sense that the weighting of the partial value of the stress index, which is determined from the pulse rate, and of the partial value of the stress index, which is determined from the heart rate variability, are optimized by trials. If there are no reasons to do so, the values c and d will be selected as 1.

It is particularly advantageous if the respectively determined stress values are not only output directly, which of course is not ruled out, but rather the current stress index SI is fed to a filter, typically to a digital low-pass filter, after the time interval Tₓ or after the predetermined number of pulse beats, in order to average over individual outliers, e.g. SI=f*SI_{z}+(1−f)*SIₓ₋₁, with f between 0,05 and 0.5, advantageously of 0.1.

According to a second aspect of the present invention there is proposed a device that is suitable for carrying out the method of the present invention, optionally including the above-mentioned advantageous embodiments.

Such a device for detecting and reporting of a stress condition of a person typically comprises an acquisition device for continuously acquiring data of the current pulse frequency and of the current heart rate variability, a processing device for continuously processing the data of the current pulse frequency and of the current heart rate variability, and a comparator device for comparing the current state function of the person thus obtained with an alert criterion.

The aforementioned elements as well as those claimed and described in the following exemplary embodiments, to be used according to the invention, are not subject to any particular conditions by way of exclusion in terms of their size, shape, use of material and technical design, with the result that the selection criteria known in the respective field of application can be used without restrictions. For carrying out the method of the present invention, the calculation of the heart rate variability can be carried out by means of conventional methods (e.g. in the time domain: RMSSD, RRinter, SDNN, or in the frequency domain LFₜₒₜ/HFₜₒₜ), wherein the method RMSSD (“Root mean square of successive differences”) is suitable for the method and device mentioned here. It should be pointed out that the method of the present invention is not intended for determining the health status or pathological status of the person, whereas the device of the present invention is not restricted in this regard.

### MODES FOR CARRYING OUT THE INVENTION

The device of the present invention comprises, according to a preferred exemplary embodiment of the invention, a measuring device for detecting the pulse rate and the values that are necessary for calculating heart rate variability. In the present case this is a pulse measuring sensor, but alternatively it can also be an electrical sensor for measuring electrical cardiographic measurement values, as well as a display device. Moreover, the device comprises an interface for the input of person-related parameters, which are particularly needed for determining the history to be used according to the invention. A key component of the device is a computing device that controls the necessary acquisition of the measurement data, processes the measurement data in the digital form needed, executes the data processing and controls the display.

In the present exemplary embodiment the heart rate variability HRV is determined by means of the RMSSD method (“Root mean square of successive differences”), but also by other methods, such as e.g. the method “SIR” based on standard deviations, the method pRR50, in which the number of consecutive RR intervals that are larger than 50 ms is determined and the value thus obtained is divided by the total number of consecutive RR intervals, or frequency-oriented methods such as, for example, the calculation via the quotient LFₜₒₜ/HFₜₒₜ of the low-frequency frequency components divided by the higher-frequency frequency components. The HRV value obtained by means of RMSSD is calculated as the square root of the sum of the squared differences between neighboring RR intervals. In this context it should be noted that for the selection of the calculation method one may use, on the one hand, pertinent recent findings of the respective technical field and of the respective application range of the method according to the present invention or of the device according to the present invention, but on the other hand it is conceivable to simply take into account practical aspects of the respective selection. In the case where the HRV values are determined by means of the RMSSD method, in the present exemplary embodiment 0 is used as tabulated value for HRVₘᵢₙ for all ages. The other minimum values used in the exemplary embodiment shown here, which refer to the pulse and to the HRV, are selected according to the following table:

Age dependent resting heart rate values

| | |
|---|---|
| Youths: 14 . . . 18 | Resting heart rate: 85 beats/minute |
| Adults: 19 . . . 65 | Resting heart rate: 70 beats/minute |
| Seniors: 65+ | Resting heart rate: 90 beats/minute |

Age dependent HRVₘₐₓ values (RMSSD)

| | |
|---|---|
| 15 . . . 20 | 47 ms |
| 21 . . . 30 | 46 ms |
| 31 . . . 40 | 40 ms |
| 41 . . . 50 | 35 ms |
| 51 . . . 60 | 30 ms |
| 61 . . . 70 | 24 ms |

according to Angelink et al: Innovationstagung FH Rapperswil 4.5.2011

In this context it should be noted that—without departing from the sense of the method of the present invention—rather different parameters of the subjects such as e.g. the gender etc. can be incorporated into the table.

According to the exemplary embodiment the normalizations are each carried out by means of a normalization value

P_{z}=Pₘᵢₙ+a*(Pₘₐₓ−Pₘᵢₙ)

HRV_{z}=HRVₘᵢₙ+b*(HRVₘₐₓ−HRVₘᵢₙ)

and the calculation of the summands of the stress value SI is each carried out according to

SI_{P}=(P_{d1}−P_{z})/(Pₘₐₓ−P_{z}) if P_{d1}>P_{z}

SI_{P}=(P_{d1}−P_{z})/(P_{z}−Pₘᵢₙ) if P_{d1}<P_{z}

SI_{HRV}=−(HRV_{d1}−HRV_{z})/(HRVₘₐₓ−HRV_{z}) if HRV_{d1}>HRV_{z}

SI_{HRV}=−(HRV_{d1}−HRV_{z})/(HRV_{z}−HRVₘᵢₙ) if HRV_{d1}<HRV_{z}

and

SI_{P}=(P_{dx}−P_{z})/(Pₘₐₓ−P_{z}) if P_{dx}>P_{z}

SI_{P}=(P_{dx}−P_{z})/(P_{z}−Pₘᵢₙ) if P_{dx}<P_{z}

SI_{HRV}=−(HRV_{dx}−HRV_{z})/(HRVₘₐₓ−HRV_{z}) if HRV_{dx}>HRV_{z}

SI_{HRV}=−(HRV_{dx}−HRV_{z})/(HRV_{z}−HRVₘᵢₙ) if HRV_{dx}<HRV_{z}

wherein a was selected as 0.25 and b as 0.5 and c and d were selected as 1. In the exemplary embodiment the current values of the stress index SI are selected after the predetermined number of pulse beats by means of a digital low-pass filter SI=f*SIₓ+(1−f)*SIₓ₋₁ calculated with f of 0.1. The device has been set up accordingly.

It should be still noted that the individual windows in which the stress index values are determined advantageously include various states of the subjects, such as e.g. lying, standing, moving—in the sense of the Conconi test—etc.

Moreover, it should be noted that even if test intervals are very widely separated in time, the adoption of the last test interval leads to a better result or more quickly to a good result than starting out with tabulated values. On the other hand, it may of course be appropriate to revert using tabulated values if the status of the subject has significantly changed in a fundamental manner.

1. A method for detecting and reporting of a stress condition of a person, wherein the method comprises the following steps:
continuously acquiring data of the current pulse frequency P and of the current heart rate variability HRV,
continuously processing the data of the current pulse frequency P and of the current heart rate variability HRV,
comparing the current state function of the person thus obtained with an alert criterion,
characterized in that
within a first time interval T₁ or across a predetermined number of pulse beats a value for the stress index SI is determined by adding a value SI_{P} for the stress index, which is obtained from a normalized average value P_{d1} of the pulse frequency in the mentioned first time interval T₁ or across the predetermined number of pulse beats, plus a value SI_{HRV}, which is obtained from a normalized average value HRV_{d1} of the heart rate variability HRV within the mentioned first time interval T₁ or across the predetermined number of pulse beats:
SI₁=c*SI_{P}+d*SI_{HRV}
wherein normalization is carried out by means of tabulated values Pₘₐₓ, Pₘᵢₙ, HRVₘₐₓ and HRVₘᵢₙ obtained from age dependent minimum and maximum pulse frequency values and HRV values, and, furthermore, the maximum and minimum values of the measured pulse frequency values and HRV values within the time interval T₁ or across the predetermined number of pulse beats are determined,
wherein T₁ lies between 100 s and 1000 s, and is preferably 300 s, or the predetermined number of pulse beats lies between 50 and 500, and is preferably 100,
in at least one further time interval Tₓ (x=2 . . . n) or across a further predetermined number of pulse beats the stress index is again determined by adding a value SI_{P} for the stress index, which is obtained from a normalized average value P_{d1} of the pulse frequency in the mentioned time interval Tₓ or across the predetermined number of pulse beats, plus a value SI_{HRV}, which is obtained from a normalized average value HRV_{d1} of the heart rate variability HRV within the mentioned time interval Tₓ or across the predetermined number of pulse beats:
SIₓ=c*SI_{P}+d*SI_{HRV}
preferably having the same length as T₁or having the same number of pulse beats,
wherein normalization is carried out by means of values Pₘₐₓ, Pₘᵢₙ, HRVₘₐₓ and HRVₘᵢₙ, wherein Pₘₐₓ and HRVₘₐₓ are selected from the larger value of Pₘₐₓ and HRVₘₐₓ determined in the previous time interval Tₓ₋₁ or across the predetermined number of pulse beats and the values of Pₘₐₓ and HRVₘₐₓ used in the previous time interval Tₓ₋₁ or across the predetermined number of pulse beats, and wherein Pₘᵢₙ and HRVₘᵢₙ are selected from the smaller value of Pₘᵢₙ and HRVₘᵢₙ determined in the previous time interval Tₓ₋₁ or across the predetermined number of pulse beats and the values of Pₘᵢₙ and HRVₘᵢₙ used in the previous time interval Tₓ₋₁ or across the predetermined number of pulse beats.

2. The method for detecting and reporting of a stress condition of a person according to claim 1, wherein normalization is each carried out by means of a normalization value
P_{z}=Pₘᵢₙ+a*(Pₘₐₓ−Pₘᵢₙ)
HRV_{z}=HRVₘᵢₙ+b*(HRVₘₐₓ−HRVₘᵢₙ)
and the calculation of the summands of the stress value SI is each carried out according to
SI_{P}=(P_{d1}−P_{z})/(Pₘₐₓ−P_{z}) if P_{d1}>P_{z}
SI_{P}=(P_{d1}−P_{z})/(P_{z}−Pₘᵢₙ) if P_{d1}<P_{z}
SI_{HRV}=−(HRV_{d1}−HRV_{z})/(HRVₘₐₓ−HRV_{z}) if HRV_{d1}>HRV_{z}
SI_{HRV}=−(HRV_{d1}−HRV_{z})/(HRV_{z}−HRVₘᵢₙ) if HRV_{d1}<HRV_{z}
and
SI_{P}=(P_{dx}−P_{z})/(Pₘₐₓ−P_{z}) if P_{dx}>P_{z}
SI_{P}=(P_{dx}−P_{z})/(P_{z}−Pₘᵢₙ) if P_{dx}<P_{z}
SI_{HRV}=−(HRV_{dx}−HRV_{z})/(HRVₘₐₓ−HRV_{z}) if HRV_{dx}>HRV_{z}
SI_{HRV}=−(HRV_{dx}−HRV_{z})/(HRV_{z}−HRVₘᵢₙ) if HRV_{dx}<HRV_{z}.

3. The method according to claim 2, wherein a is selected as 0.25 and b as 0.5.

4. The method according to claim 1, wherein c and d are selected as 1.

5. The method according to claim 1, wherein the current stress index SI is selected after the time interval Tₓ or after the predetermined number of pulse beats by means of a digital low-pass filter SI=f*SIₓ+(1−f)*SIₓ₋₁ with f between 0.05 and 0.5, preferably equal to 0.1.

6. The method according to claim 1, wherein the time intervals or the times during which a predetermined number of pulse beats are measured, overlap.

7. The method according to claim 1, wherein the time intervals or the times in which a predetermined number of pulse beats are measured, have a fixed or variable distance between each other.

8. A device for detecting and reporting of a stress condition of a person, comprising:
an acquisition device for continuously acquiring data of the current pulse frequency and of the current heart rate variability,
a processing device for continuously processing the data of the current pulse frequency and of the current heart rate variability, and
a comparator device for comparing the obtained current state function of the person with an alert criterion
characterized in that
the processing device is configured in such manner that within a first time interval T₁ or across a predetermined number of pulse beats a value for the stress index SI is determined by adding a value SI_{P} for the stress index, which is obtained from a normalized average value P_{d1} of the pulse frequency in the mentioned first time interval T₁ or across the predetermined number of pulse beats, plus a value SI_{HRV}, which is obtained from a normalized average value HRV_{d1} of the heart rate variability HRV within the mentioned first time interval T₁ or across the predetermined number of pulse beats:
SI₁=c*SI_{P}+d*SI_{HRV}
wherein normalization is carried out by means of tabulated values Pₘₐₓ, Pₘᵢₙ, HRVₘₐₓ and HRVₘᵢₙ obtained from age dependent minimum and maximum pulse frequency values and HRV values, and, furthermore, the maximum and minimum values of the measured pulse frequency values and HRV values within the time interval T₁ or across the predetermined number of pulse beats are determined,
wherein T₁ lies between 100 s and 1000 s, and is preferably 300 s, or the predetermined number of pulse beats lies between 50 and 500, and is preferably 100,
in at least one further time interval Tₓ (x=2 . . . n) or across a further predetermined number of pulse beats the stress index is again determined by adding a value SI_{P} for the stress index, which is obtained from a normalized average value P_{d1} of the pulse frequency in the mentioned time interval Tₓ or across the predetermined number of pulse beats, plus a value SI_{HRV}, which is obtained from a normalized average value HRV_{d1} of the heart rate variability HRV within the mentioned time interval Tₓ or across the predetermined number of pulse beats:
SIₓ=c*SI_{P}+d*SI_{HRV}
preferably having the same length as T₁ or having the same number of pulse beats,
wherein normalization is carried out by means of values Pₘₐₓ, Pₘᵢₙ, HRVₘₐₓ and HRVₘᵢₙ, wherein Pₘₐₓ and HRVₘₐₓ are selected from the larger value of Pₘₐₓ and HRVₘₐₓ determined in the previous time interval Tₓ₋₁ or across the predetermined number of pulse beats and the values of Pₘₐₓ and HRVₘₐₓ used in the previous time interval Tₓ₋₁ or across the predetermined number of pulse beats, and wherein Pₘᵢₙ and HRVₘᵢₙ are selected from the smaller value of Pₘᵢₙ and HRVₘᵢₙ determined in the previous time interval Tₓ₋₁ or across the predetermined number of pulse beats and the values of Pₘᵢₙ and HRVₘᵢₙ used in the previous time interval Tₓ₋₁ or across the predetermined number of pulse beats.

9. The device according to claim 8, wherein the device is configured in such manner that the method can be carried out according to claim 2.

10. The device according to claim 8, wherein a is selected as 0.25 and b as 0.5 and/or wherein c and d are selected as 1, and/or wherein the current stress index SI is selected after the time interval Tₓ or after the predetermined number of pulse beats by means of a digital low-pass filter SI=f*SIₓ+(1−f)*SIₓ₋₁
with f between 0.05 and 0.5, preferably 0.1.

11. The device according to claim 9, wherein a is selected as 0.25 and b as 0.5 and/or wherein c and d are selected as 1, and/or wherein the current stress index SI is selected after the time interval Tₓ or after the predetermined number of pulse beats by means of a digital low-pass filter SI=f*SIₓ+(1−f)*SIₓ₋₁
with f between 0.05 and 0.5, preferably 0.1.

12. The method according to claim 2, wherein c and d are selected as 1.

13. The method according to claim 3, wherein c and d are selected as 1.

14. The method according to claim 2, wherein the current stress index SI is selected after the time interval Tₓ or after the predetermined number of pulse beats by means of a digital low-pass filter SI=f*SIₓ+(1−f)*SIₓ₋₁ with f between 0.05 and 0.5, preferably equal to 0.1.

15. The method according to claim 3, wherein the current stress index SI is selected after the time interval Tₓ or after the predetermined number of pulse beats by means of a digital low-pass filter SI=f*SIₓ+(1−f)*SIₓ₋₁ with f between 0.05 and 0.5, preferably equal to 0.1.

16. The method according to claim 4, wherein the current stress index SI is selected after the time interval Tₓ or after the predetermined number of pulse beats by means of a digital low-pass filter SI=f*SIₓ+(1−f)*SIₓ₋₁ with f between 0.05 and 0.5, preferably equal to 0.1.

17. The method according to claim 2, wherein the time intervals or the times during which a predetermined number of pulse beats are measured, overlap.

18. The method according to claim 3, wherein the time intervals or the times during which a predetermined number of pulse beats are measured, overlap.

19. The method according to claim 4, wherein the time intervals or the times during which a predetermined number of pulse beats are measured, overlap.

20. The method according to claim 5, wherein the time intervals or the times during which a predetermined number of pulse beats are measured, overlap.

21. The method according to claim 2, wherein the time intervals or the times in which a predetermined number of pulse beats are measured, have a fixed or variable distance between each other.

22. The method according to claim 3, wherein the time intervals or the times in which a predetermined number of pulse beats are measured, have a fixed or variable distance between each other.

23. The method according to claim 4, wherein the time intervals or the times in which a predetermined number of pulse beats are measured, have a fixed or variable distance between each other.

24. The method according to claim 5, wherein the time intervals or the times in which a predetermined number of pulse beats are measured, have a fixed or variable distance between each other.
